(19) United States
(12) Patent Application Publication
(10) Pub. No.: US 2021/0319909 A1
(43) Pub. Date: Oct. 14, 2021

(54) METHODS AND SYSTEMS FOR INHIBITING THE SPREAD OF A PATHOGEN

(71) Applicant: Daon Holdings Limited, West Bay (KY)

(72) Inventor: Thomas GRISSEN, Fairfax, VA (US)

(21) Appl. No.: 16/847,003

(22) Filed: Apr. 13, 2020

(51) Int. Cl. G16H 50/80; G16H 10/60; G16H 70/60; A61B 5/00; H04L 29/08

(52) U.S. Cl. CPC G16H 50/80; G16H 10/60; G06Q 50/26; A61B 5/4842; H04L 67/18; G16H 70/60

(57) ABSTRACT

A method for inhibiting the spread of a pathogen is provided that includes storing, using a computer, location data of computing devices. Each computing device is associated with a respective user. The method also includes receiving data indicating one of the users contracted a pathogen, and comparing, using the computer, the location data of the computing device associated with the pathogenic user against the location data of the other computing devices to determine whether the computing device of the pathogenic user was within a distance of at least one other computing device within a period of time. When at least one other computing device was within the distance of the computing device associated with the pathogenic user, the user associated with the at least one other computing device is identified and notified.

### BACKGROUND OF THE INVENTION

This invention relates generally to health and welfare of people, and more particularly to methods and systems for inhibiting the spread of a pathogen between people.

Some pathogens are known to be highly contagious while others are less contagious. People may transmit pathogens between each other by shaking hands or otherwise coming into contact with each other. Some pathogens may remain live on exposed surfaces for several hours and before expiring may be touched by a person who may then be able to transmit the pathogen to others. Some pathogens are more lethal than others. Quickly spreading pathogens can result in increased fatalities and significant economic deterioration.

Current techniques for identifying people who may have been in contact with a person infected with a pathogen are limited to interviewing the infected person about his or her movements and personal interactions. While people may be very cooperative and try to provide the best information, it is difficult for one to remember all his or her movements and to identify all the people one has interacted with over a specified period of time. Thus, current methods are not as accurate as desired. Additionally, it may take valuable time to contact and interview the identified people which increases costs and in urgent circumstances fatalities.

### BRIEF DESCRIPTION OF THE INVENTION

In one aspect, a method for inhibiting the spread of a pathogen is provided that includes storing, using a computer, location data of computing devices. Each computing device is associated with a respective user. The method also includes receiving data indicating one of the users contracted a pathogen, and comparing, using the computer, the location data of the computing device associated with the pathogenic user against the location data of the other computing devices to determine whether the computing device of the pathogenic user was within a distance of at least one other computing device within an period of time. When at least one other computing device was within the distance of the computing device associated with the pathogenic user, the user associated with the at least one other computing device is identified and notified.

In another aspect, a computer for inhibiting the spread of a pathogen is provided that includes a processor and a memory. The memory is configured to store location data of computing devices. Each computing device is associated with a respective user. The computer is associated with a network and the memory is in communication with the processor and has instructions stored therein which, when read and executed by the processor, cause the computer to receive data indicating one of the users contracted a pathogen.

Moreover, the instructions, when read and executed by the processor, cause the computer to compare the location data of the computing device associated with the pathogenic user against the location data of the other computing devices to determine whether the computing device of the pathogenic user was within a distance of at least one other computing device within a period of time. When at least one other computing device was within the distance of the computing device associated with the pathogenic user, the instructions, when read and executed by the processor, cause the computer to identify the user associated with the at least one other computing device and notify the identified user.

In yet another aspect, a non-transitory computer-readable recording medium is provided that is included in a computer having a computer program recorded thereon for inhibiting the spread of a pathogen. The computer program is comprised of instructions, which when read and executed by the computer, cause the computer to store location data of computing devices. Each computing device is associated with a respective user. The instructions, which when read and executed by the computer, also cause the computer to receive data indicating one of the users contracted a pathogen.

Moreover, the instructions, which when read and executed by the computer, cause the computer to compare the location data of the computing device associated with the pathogenic user against the location data of the other computing devices to determine whether the computing device of the pathogenic user was within a distance of at least one other computing device within a period of time. When at least one other computing device was within the distance of the computing device associated with the pathogenic user, the instructions, which when read and executed by the computer, cause the computer to identify the user associated with the at least one other computing device and notify the identified user.

### BRIEF DESCRIPTION OF THE DRAWINGS

FIG. 1 is a schematic diagram illustrating an example system for inhibiting the spread of a pathogen;

FIG. 2 is a schematic diagram illustrating an example architecture of an example computer device used for inhibiting the spread of pathogens; and

FIG. 3 is a flowchart illustrating an example method of inhibiting the spread of pathogens.

### DETAILED DESCRIPTION OF THE INVENTION

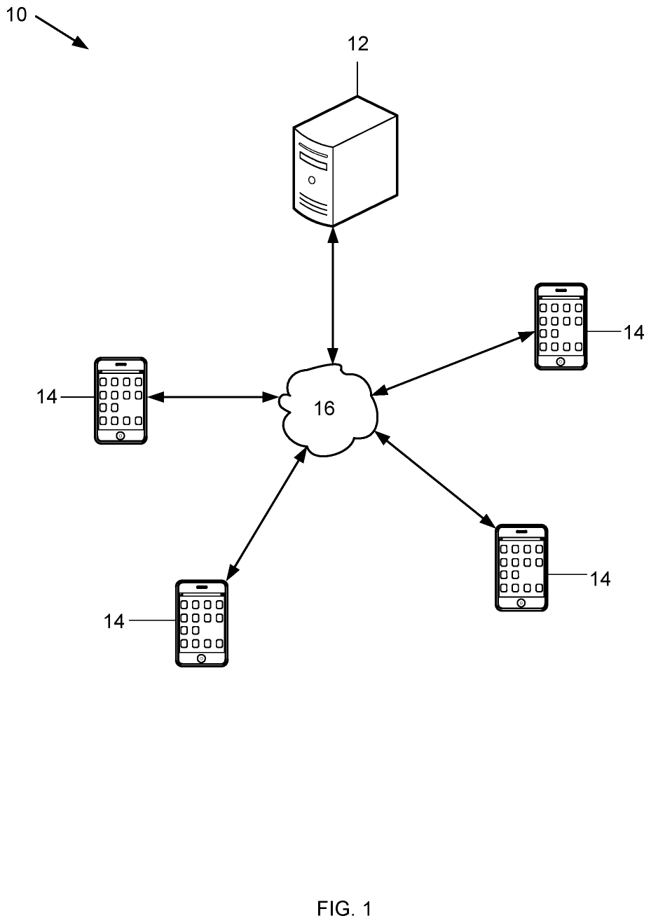

FIG. 1 is a schematic diagram illustrating an example system 10 for inhibiting the spread of a pathogen. The system 10 includes a computer 12, computing devices 14, and a network 16. The computer 12 and the computing device 14 communicate over the network 16. Although four computing devices 14 are included in the example system 10, it is contemplated that any number of computing devices 14 may be included in the system 10. As used herein, a pathogen is considered to be a living microorganism that causes a disease. Examples of pathogens include, but are not limited to, microorganisms, bacteria, and viruses.

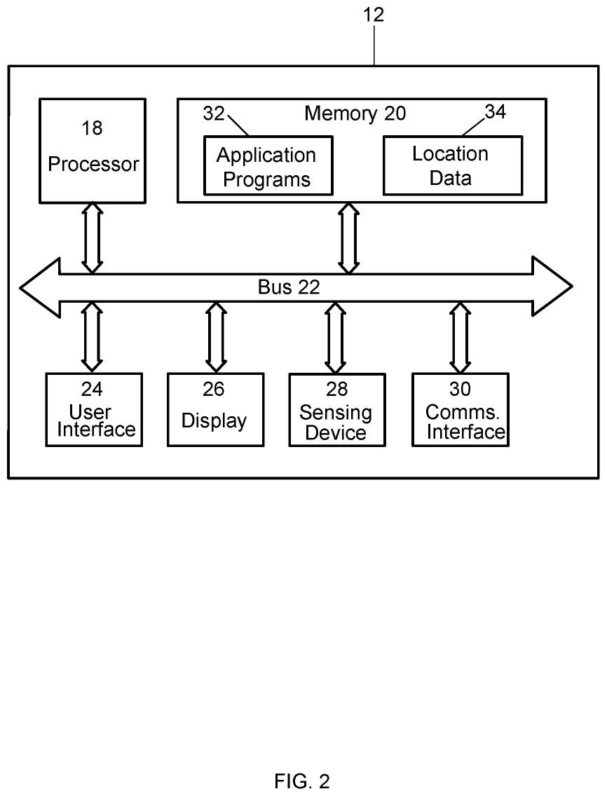

FIG. 2 is a schematic diagram illustrating an example architecture of an example computer 12 used for inhibiting the spread of pathogens. The computer 12 includes components such as, but not limited to, one or more processors 18, a memory 20, a bus 22, a user interface 24, a display 26, a sensing device 28, and a communications interface 30. General communication between the components in the computer 12 is provided via the bus 22.

The computer 12 may be a network server, a personal computer WC), or any other computing device that can store data and communicate over networks, and is capable of performing the functions described herein.

The processor 18 executes instructions, or computer programs, stored in the memory 20. As used herein, the term processor is not limited to just those integrated circuits referred to in the art as a processor, but broadly refers to a computer, a microcontroller, a microcomputer, a programmable logic controller, an application specific integrated circuit, and any other programmable circuit capable of executing at least a portion of the functions and/or methods described herein. The above examples are not intended to limit in any way the definition and/or meaning of the term “processor.”

As used herein, the term “computer program” is intended to encompass an executable program that exists permanently or temporarily on any non-transitory computer-readable recordable medium that causes the computer 12 to perform at least a portion of the functions and/or methods described herein. Application programs 32, also known as applications, are computer programs stored in the memory 20. Application programs 32 include, but are not limited to, an operating system, an Internet browser application, enrolment applications, applications that compare location data of different computing devices and calculate relationships between the location data, and any special computer program that manages the relationship between application software and any suitable variety of hardware that helps to make-up a computer system or computing environment.

The memory 20 may be any non-transitory computer-readable recording medium used to store data including, but not limited to, computer programs 32 and location data 34 of computing devices. The location data 34 is typically Global Positioning Coordinate (GPS) data. Alternatively, the location data 34 may be any data that facilitates calculating relationships between past locations of computing devices 14.

Non-transitory computer-readable recording media may be any tangible computer-based device implemented in any method or technology for short-term and long-term storage of information or data. Moreover, the non-transitory computer-readable recording media may be implemented using any appropriate combination of alterable, volatile or non-volatile memory or non-alterable, or fixed, memory. The alterable memory, whether volatile or non-volatile, can be implemented using any one or more of static or dynamic RAM (Random Access Memory), a floppy disc and disc drive, a writeable or re-writeable optical disc and disc drive, a hard drive, flash memory or the like. Similarly, the non-alterable or fixed memory can be implemented using any one or more of ROM (Read-Only Memory), PROM (Programmable Read-Only Memory), EPROM (Erasable Programmable Read-Only Memory), EEPROM (Electrically Erasable Programmable Read-Only Memory), an optical ROM disc, such as a CD-ROM or DVD-ROM disc, and disc drive or the like. Furthermore, the non-transitory computer-readable recording media may be implemented as smart cards, SIMs, any type of physical and/or virtual storage, or any other digital source such as a network or the Internet from which a computing device can read computer programs, applications or executable instructions.

The user interface 24 and the display 26 allow interaction between a user and the computer 12. The display 26 may include a visual display or monitor that displays information to a user. For example, the display 26 may be a Liquid Crystal Display (LCD), active matrix display, plasma display, or cathode ray tube (CRT). The user interface 24 may include a keypad, a keyboard, a mouse, an illuminator, a signal emitter, a microphone, and/or speakers.

Moreover, the user interface 24 and the display 26 may be integrated into a touch screen display. Accordingly, the display may also be used to show a graphical user interface, which can display various data and provide “forms” that include fields that allow for the entry of information by the user. Touching the screen at locations corresponding to the display of a graphical user interface allows the user to interact with the computer 12 to enter data, change settings, control functions, etc. Consequently, when the touch screen is touched, the user interface 24 communicates this change to the processor 18, and settings can be changed or user entered information can be captured and stored in the memory 20.

The sensing device 28 may include Radio Frequency Identification (RFID) components or systems for receiving information from other devices. The sensing device 28 may alternatively, or additionally, include components with Bluetooth, Near Field Communication (NFC), infrared, or other similar capabilities. The computer 12 may alternatively not include the sensing device 28.

The communications interface 30 provides the computer 12 with two-way data communications. Moreover, the communications interface 30 enables the computer 12 to conduct wireless communications such as cellular telephone calls and to wirelessly access the Internet over the network 16. By way of example, the communications interface 30 may be a digital subscriber line (DSL) card or modem, an integrated services digital network (ISDN) card, a cable modem, or a telephone modem to provide a data communication connection to a corresponding type of telephone line. As another example, the communications interface 30 may be a local area network (LAN) card (e.g., for Ethemet™ or an Asynchronous Transfer Model (ATM) network) to provide a data communication connection to a compatible LAN. As yet another example, the communications interface 30 may be a wire or a cable connecting the computer 12 with a LAN, or with accessories such as, but not limited to, other computing devices. Further, the communications interface 30 may include peripheral interface devices, such as a Universal Serial Bus (USB) interface, a PCMCIA (Personal Computer Memory Card International Association) interface, and the like. Thus, it should be understood the communications interface 30 may enable the computer 12 to conduct any type of wireless or wired communications such as, but not limited to, accessing the Internet. Although the computer 12 includes a single communications interface 30, the computer 12 may alternatively include multiple communications interfaces 30.

The communications interface 30 also allows the exchange of information across the network 16. The exchange of information may involve the transmission of radio frequency (RF) signals through an antenna (not shown). Moreover, the exchange of information may be between the computer 12 and any other computing devices 14 capable of communicating over the network 16.

The computer 12 may receive location data 34 collected and transmitted by computing devices 14. The computer 12 may compare the location data 34 collected by different computing devices 14 to determine whether or not a computing device 14 was proximate another computing device 14 within a period of time. The computer 12 is able to calculate the time, duration, and location where a computing device 14 was proximate another computing device 14. The user associated with a computing device 14 is considered to be in the same location as his or her computing device 14.

Should a user contract a pathogen, the location data 34 collected by the computing device 14 of the pathogenic user may be compared against the stored location data 34 to facilitate identifying users who were proximate the pathogenic user within a specified period of time. Proximate may be quantified by specifying a distance according to criteria established by the United States Centers for Disease Control (C.D.C.). For example, a user may be considered to have been proximate a pathogenic user if he or she was within six feet of the pathogenic user. Alternatively, any other distance or standard established by the C.D.C. or any other governmental agency or commercial entity may be used.

The specific period of time may depend on the viability of a pathogen outside of a host organism and may also be established by the C.D.C or other governmental agency or commercial entity. The specified period of time may be within a range of between about seven and fourteen days. However, the specified period of time may be any period of time that enables identifying users who may have been in contact with a pathogenic user and may have contracted the pathogen.

The computer 12 may be operated by an entity offering a service which facilitates notifying users regarding whether he or she has been in close proximity to another user who has contracted a pathogen. The service may be voluntary and data associated with users is maintained as anonymous. As used herein, users are people who are associated with a computing device 14 enrolled in the service. Typically, enrolling in the service allows location data 34 collected by a user's computing device 14 to be transmitted to the computer 12 for storage therein and to be anonymously compared against stored location data 34 of other enrolled computing devices 14.

The computing devices 14 typically include components similar to the components described herein for the computer 12. Additionally, the computing device 14 may include a gyroscope and one or more accelerometers that generate data regarding rotation and translation of the computing devices 14. Each computing device 14 includes a location services application that captures location data 34 of the computing device 14. The location data 34 is typically captured as Global Positioning Coordinates (GPS) and is transmitted to the computer 12 for storage therein. However, the location data 34 may be any data that facilitates calculating relationships between locations of computing devices 14. Additionally, each computing device 14 is capable of performing at least the functions described herein.

One example of a computing device 14 is a smart phone. Other examples include, but are not limited to, a cellular phone, a tablet computer, a phablet computer, a laptop computer, and any type of device having wired or wireless networking capabilities such as a personal digital assistant (PDA). Each computing device 14 is typically portable and may be associated with any individual or with any type of entity including, but not limited to, commercial and non-commercial entities. Additionally, the computing devices 14 may alternatively be referred to as computer systems or information systems.

The network 16 may be a 5G communications network. Alternatively, the network 16 may be any wireless network including, but not limited to, 4G, 3G, Wi-Fi, Global System for Mobile (GSM), Enhanced Data for GSM Evolution (EDGE), and any combination of a LAN, a wide area network (WAN) and the Internet. The network 16 may also be any type of wired network or a combination of wired and wireless networks.

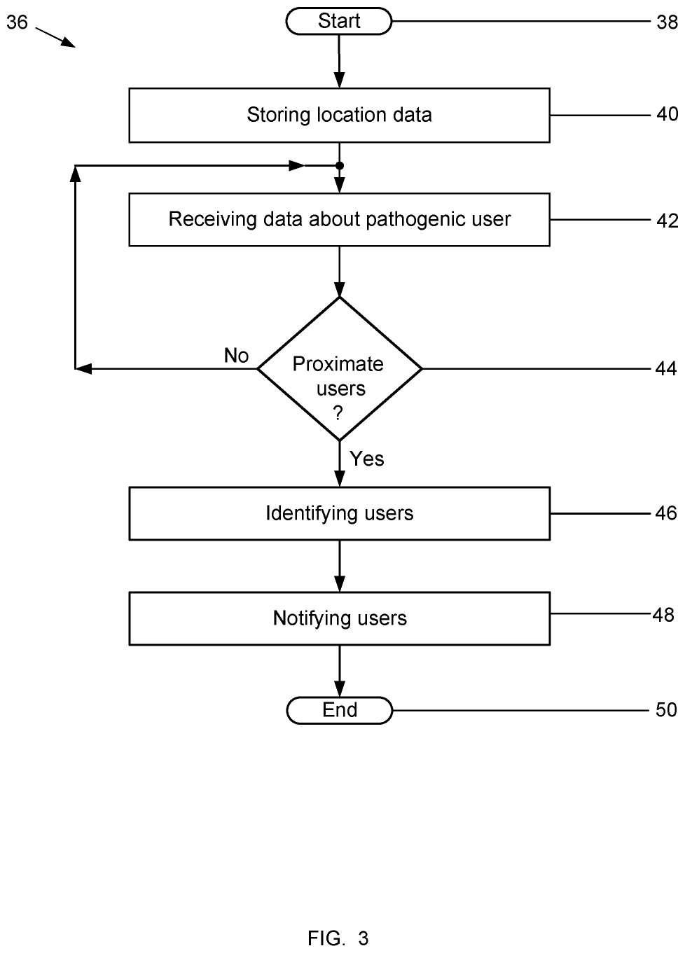

FIG. 3 is flowchart 36 illustrating an example method of inhibiting the spread of pathogens. The methods starts 38 by storing 40 in the computer 12 location data 34 collected by computing devices 14. Each of the computing devices 14 is associated with a respective user who voluntarily enrolled in a service offered by the operator of the computer 12. Next, the computer 12 continues by receiving 42 data indicating that one of the users contracted a pathogen. In response to receiving 40 the data, the computer 12 continues by comparing 44 the location data 34 of the computing device 14 associated with the pathogenic user against the location data 34 stored therein to determine whether or not the computing device 14 of the pathogenic user was proximate at least one other computing device 14 within a period of time. In this example method, the computing devices 14 are considered to be proximate if they are within six feet of each other and the period of time is two weeks.

When none of the other computing devices 14 was proximate the computing device 14 associated with the pathogenic user within the previous two weeks, processing continues by waiting to receive 42 data about another pathogenic user. However, when at least one of the other computing devices 14 was proximate the computing device 14 associated with the pathogenic user within the previous two weeks, the computer 12 continues by identifying 46 the users associated with the other computing devices 14 and notifying 48 the identified users. The computer 12 may notify the users by transmitting an email message or an SMS text message to the computing device 14 associated with the respective user. Next, processing ends 50.

The example methods described herein may be conducted entirely by the computer 12, or partly on the computer 12 and partly on computing devices 14 operable to communicate with the computer 12 over the network 16. It is contemplated that the example methods may be conducted on any combination of computers, computer systems, and computing devices. Furthermore, data described herein as being stored in the computer 12 may alternatively be stored in any computer system operable to communicate with the computer 12 over the network 16. Additionally, the example methods described herein may be implemented with any number and organization of computer program components. Thus, the methods described herein are not limited to specific computer-executable instructions. Alternative example methods may include different computer-executable instructions or components having more or less functionality than described herein.

In example embodiments, the above-described methods and systems for inhibiting the spread of a pathogen enhance the speed and accuracy of identifying people who may have been exposed to individuals who have tested positive for a pathogen. More specifically, in one example embodiment, after receiving data about a pathogenic user a computer compares location data of the computing device associated with the pathogenic user against the location data of other computing devices. If any of the other computing devices is judged to have been proximate the pathogenic user's computing device within a specified period of time, the computer identifies the users of the other computing devices and transmits a notification to the users.

As a result, in each example embodiment, the speed and accuracy of identifying people who may have been exposed to individuals who have tested positive for a pathogen are facilitated to be enhanced. People who have been exposed to individuals who have tested positive may implement measures to inhibit spread of the pathogen. For example, such individuals may quarantine for a period of time or seek medical attention. As a result, in each example embodiment the monetary costs, in medical terms as well as general economic terms, borne by society as a result of deadly pathogens are facilitated to be reduced. The number of fatalities suffered by deadly pathogens are also facilitated to be reduced.

The example methods and systems for inhibiting the spread of a pathogen described above should not be considered to imply a fixed order for performing the method steps. Rather, the method steps may be performed in any order that is practicable, including simultaneous performance of at least some steps. Moreover, the method steps may be performed in real time or in near real time. It should be understood that, for any process described herein, there can be additional, fewer, or alternative steps performed in similar or alternative orders, or in parallel, within the scope of the various embodiments, unless otherwise stated. Furthermore, the invention is not limited to the embodiments of the methods described above in detail. Rather, other variations of the methods may be utilized within the spirit and scope of the claims.

What is claimed is:

1. A method for inhibiting the spread of a pathogen comprising the steps of:
storing location data of computing devices in a computer, each computing device being associated with a respective user;
receiving, using the computer, data indicating one of the users contracted a pathogen;
comparing, using the computer, the location data of the computing device associated with the pathogenic user against the location data of the other computing devices to determine whether the computing device of the pathogenic user was within a distance of at least one other computing device within a period of time;
when at least one other computing device was within the distance of the computing device associated with the pathogenic user, identifying the user associated with the at least one other computing device; and
notifying the identified user.

2. The method for inhibiting the spread of a pathogen according to claim 1, wherein the distance is defined according to criteria established by the United States Center for Disease Control.

3. The method for inhibiting the spread of a pathogen according to claim 1, wherein the distance is less than or equal to six feet from the at least one other computing device.

4. The method for inhibiting the spread of a pathogen according to claim 1, wherein the the period of time is fourteen days.

5. The method for inhibiting the spread of a pathogen according to claim 1 further comprising the steps of:
capturing the location data of the computing devices; and
transmitting the captured location data to the computer.

6. The method for inhibiting the spread of a pathogen according to claim 1 further comprising the step of voluntarily enrolling the computing device of a user in a tracking service offered by an entity operating the computer.

7. The method for inhibiting the spread of a pathogen according to claim 1 further comprising the step of invoking a location services operation in the computing devices, wherein the location services operation causes the computing device to capture location data.

8. A computer for inhibiting the spread of a pathogen comprising:
a processor; and
a memory configured to store location data of computing devices, each computing device being associated with a respective user, said computer being associated with a network and said memory being in communication with said processor and having instructions stored therein which, when read and executed by said processor, cause said computer to:
receive data indicating one of the users contracted a pathogen;
compare the location data of the computing device associated with the pathogenic user against the location data of the other computing devices to determine whether the computing device of the pathogenic user was within a distance of at least one other computing device within a period of time;
when at least one other computing device was within the distance of the computing device associated with the pathogenic user, identify the user associated with the at least one other computing device; and
notify the identified user.

9. The computer for inhibiting the spread of a pathogen in accordance with claim 8, wherein the distance is defined according to criteria established by the United States Center for Disease Control.

10. The computer for inhibiting the spread of a pathogen in accordance with claim 8, wherein the distance is less than or equal to six feet from the at least one other computing device.

11. The computer for inhibiting the spread of a pathogen in accordance with claim 8, wherein the period of time is fourteen days.

12. A non-transitory computer-readable recording medium included in a computer having a computer program recorded thereon for inhibiting the spread of a pathogen, the computer program being comprised of instructions, which when read and executed by the computer, cause the computer to:
store location data of computing devices, each computing device being associated with a respective user;
receive data indicating one of the users contracted a pathogen;
compare the location data of the computing device associated with the pathogenic user against the location data of the other computing devices to determine whether the computing device of the pathogenic user was within a distance of at least one other computing device within a period of time;
when at least one other computing device was within the distance of the computing device associated with the pathogenic user, identify the user associated with the at least one other computing device; and
notify the identified user.

13. The non-transitory computer-readable recording medium according to claim 12, wherein the distance is defined according to criteria established by the United States Center for Disease Control.

14. The non-transitory computer-readable recording medium according to claim 12, wherein the distance is less than or equal to six feet from the at least one other computing device.

15. The non-transitory computer-readable recording medium according to claim 12, wherein the period of time is fourteen days.
